(19) United States
(12) Patent Application Publication
(10) Pub. No.: US 2026/0120822 A1
(43) Pub. Date: Apr. 30, 2026

(54) COMPUTER-READABLE RECORDING MEDIUM, INFORMATION PROCESSING METHOD, AND INFORMATION PROCESSING DEVICE

(71) Applicant: Fujitsu Limited, Kawasaki-shi (JP)

(72) Inventor: Akito FUJII, Fukuoka (JP)

(73) Assignee: Fujitsu Limited, Kawasaki-shi (JP)

(21) Appl. No.: 19/367,903

(22) Filed: Oct. 24, 2025

(30) Foreign Application Priority Data
Oct. 28, 2024 (JP) 2024-189189

(51) Int. Cl. G16C 60/00; G16C 20/80

(52) U.S. Cl. CPC G16C 60/00; G16C 20/80

(57) ABSTRACT

An information processing device includes a control unit. The control unit executes a process including acquiring experimental data including an experimental condition of each of a plurality of times of experiments and an observed value under the experimental condition, and generating graph data indicating a topological structure of a geometric figure representing a correspondence between the experimental condition and the observed value in a multidimensional space with each of the experimental condition and the observed value being a dimension.

### CROSS-REFERENCE TO RELATED APPLICATION

This application is based upon and claims the benefit of priority of the prior Japanese Patent Application No. 2024-189189, filed on Oct. 28, 2024, the entire contents of which are incorporated herein by reference.

### FIELD

The embodiments discussed herein are related to a computer-readable recording medium, an information processing method, and an information processing device.

### BACKGROUND

In condensed matter physics, one of the indices used for understanding physical properties (electron states, crystal structures, and the like) of metals is a Fermi surface that exists in wave number space and that has a geometric structure. The topology of this Fermi surface is related to physical properties of metals, so that, for analysis of the physical properties, the topological structure of the Fermi surface under different experimental conditions (for example, intensity of electron energy applied to the metal surface) has conventionally been studied experimentally. The related technologies are described, for example, in: U.S. Patent Application Publication No. 2022/0106334.

### SUMMARY

According to an aspect of an embodiment, an information processing device includes a control unit. The control unit executes a process including acquiring experimental data including an experimental condition of each of a plurality of times of experiments and an observed value under the experimental condition, and generating graph data indicating a topological structure of a geometric figure representing a correspondence between the experimental condition and the observed value in a multidimensional space with each of the experimental condition and the observed value being a dimension.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a block diagram illustrating an example functional configuration of an information processing device according to an embodiment;

FIG. 2 is a diagram illustrating mathematical description of a Reeb graph;

FIG. 3 is a flowchart illustrating example operation of the information processing device according to the embodiment;

FIG. 4 is a flowchart illustrating an example process of generating the Reeb graph;

FIG. 5 is an explanatory diagram describing example display of the Reeb graph and a Fermi surface;

FIG. 6 is an explanatory diagram exemplifying the Fermi surface for each experimental condition; and

FIG. 7 is an explanatory diagram describing an example computer configuration.

### DESCRIPTION OF EMBODIMENTS

Unfortunately, there is a problem in that it is difficult to follow how the topology of the Fermi surface changes (the topological structure of the Fermi surface) by changing the experimental conditions throughout the experiment on the basis of a large number of experimental conditions existing and observed values acquired under those experimental conditions.

Preferred embodiments of the present invention will be explained with reference to accompanying drawings. In the embodiments, constituents having the same function are denoted by the same reference sign, and overlapping description is omitted. Note that the information processing computer program, the information processing method, and the information processing device described in the following embodiments are merely examples and do not limit the embodiments. Each of the following embodiments may be combined as appropriate to the extent to which inconsistency does not arise.

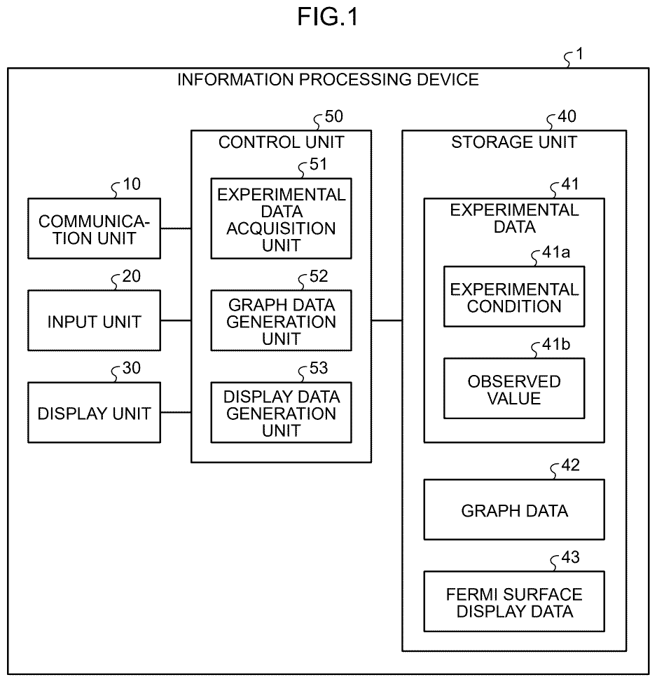

FIG. 1 is a block diagram illustrating an example functional configuration of an information processing device according to an embodiment. The information processing device 1 illustrated in FIG. 1 is a device that performs data analysis of experimental data 41 including experimental conditions 41a of a plurality of times of experiments and an observed value 41b observed under each of the experimental conditions 41a. For example, a personal computer (PC) or the like can be applied to the information processing device 1.

Note that, in this embodiment, as an example, experimental data 41 in physical property experiments performed a plurality of times for understanding physical properties (an electron state, a crystal structure, and the like) of a predetermined metal is an object to be analyzed with the information processing device 1. In the physical property experiments, with the intensity of electron energy applied to the surface of the metal as the experimental conditions 41a, information related to a Fermi surface in wave number space (an iso-surface in wave number space) is acquired as the observed values 41b. In the physical property experiments, as results of the plural times of experiments with different experimental conditions 41a, the experimental data 41 including the observed value 41b under each experimental condition 41a is acquired.

Note that the experimental data 41 being an object to be analyzed with the information processing device 1 is not limited to the acquisition in the above-described physical property experiments. For example, the experimental data 41 being an object to be analyzed with the information processing device 1 may be data in fluid experiments with the speed of a fluid as the experimental conditions 41a and streamlines as the observed values 41b. Alternatively, the experimental data 41 may be data in chemical experiments or biotic experiments with temperature, humidity, and the like as the experimental conditions 41a and the amounts of a predetermined product generated chemically/biotically and the like as the observed values 41b.

As illustrated in FIG. 1, the information processing device 1 includes a communication unit 10, an input unit 20, a display unit 30, a storage unit 40, and a control unit 50.

The communication unit 10 executes data communications with an external device or the like via a network under control at the control unit 50. For example, the communication unit 10 may acquire the experimental data 41 from an experiment apparatus (not illustrated) connected via the network.

The input unit 20 receives operation from a user. For example, the input unit 20 may acquire the experimental data 41 through input operation by the user.

The display unit 30 displays a result of a process at the control unit 50, such as graph data 42 and Fermi surface display data 43, under control at the control unit 50.

The storage unit 40 stores therein various pieces of data, such as the experimental data 41, the graph data 42, and the Fermi surface display data 43. For example, the storage unit 40 is implemented by a memory or the like.

The graph data 42 is data related to a graph acquired by the data analysis based on the experimental data 41 (See below for details). The Fermi surface display data 43 is display data that indicates the experimental condition 41a and the observed value 41b corresponding to a specific point on the graph of the graph data 42. To be specific, the Fermi surface display data 43 is data that displays a Fermi surface in the wave number space under the experimental condition 41a corresponding to a specific point on the graph of the graph data 42.

The control unit 50 includes an experimental data acquisition unit 51, a graph data generation unit 52, and a display data generation unit 53. For example, the control unit 50 is implemented by a processor.

The experimental data acquisition unit 51 is a processing unit that acquires the experimental data 41 via the communication unit 10 or the input unit 20. To be specific, the experimental data acquisition unit 51 acquires the experimental data 41 including the experimental condition 41a of each of the plural times of experiments and the observed value 41b observed under that experimental condition 41a through data input from the experiment apparatus, the input operation by the user, or the like. The experimental data acquisition unit 51 stores the acquired experimental data 41 in the storage unit 40.

The graph data generation unit 52 is a processing unit that generates the graph data 42 related to a graph representing correspondences between a plurality of the experimental conditions 41a and the observed values 41b included in the experimental data 41 on the basis of the experimental data 41. To be specific, the graph data generation unit 52 generates the graph data 42 of a graph indicating the topological structure of a geometric figure representing the correspondences between the experimental conditions 41a in the plural times of experiments and the observed values 41b in a multidimensional space with each of the experimental conditions 41a and the observed values 41b being a dimension.

The graph data generation unit 52 stores the generated graph data 42 together with a pair of the experimental data 41 (the experimental condition 41a and the observed value 41b) corresponding to each point on the graph, in the storage unit 40. This allows the control unit 50 to read out and display the graph data 42 on the display unit 30 to present a Reeb graph G1 to the user. The display data generation unit 53 can generate the Fermi surface display data 43 on the basis of the pair of the experimental data 41 (the experimental condition 41a and the observed value 41b) corresponding to each point on the graph (See below for details).

Such graphs indicating the topological structure include a Reeb graph. The graph data generation unit 52 generates the graph data 42 of a Reeb graph indicating the correspondences between the experimental conditions 41a and the observed values 41b included in the experimental data 41 on the basis of the experimental data 41. Note that the graph data 42 generated by the graph data generation unit 52 is not limited to the above-described Reeb graph and may be data related to other graphs, such as a contour tree, as long as it is a similar graph indicating the topological structure.

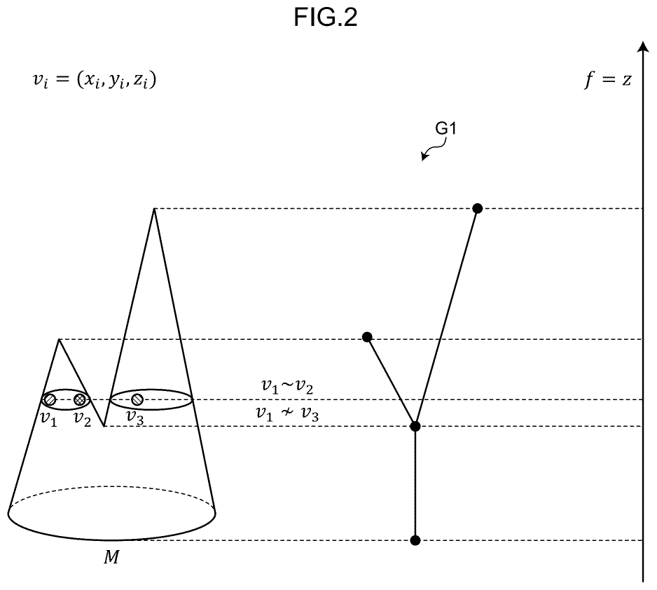

FIG. 2 is a diagram illustrating mathematical description of the Reeb graph. FIG. 2 exemplifies, given a continuous space M and a continuous function f: M→R, equivalence relations defined in the continuous space M. As illustrated in FIG. 2, the equivalence relation v˜u is defined as the connected component of f⁻¹ (f(v)) and the connected component of f⁻¹ (f(u)) being the same. For example, taking v₁, v₂, and v₃ in the continuous space M illustrated in FIG. 2 as an example, v₁ and v₂ are in an equivalence relation v₁˜v₂. On the other hand, v₁ and v₃, and v₂ and v₃ are not in equivalence relations. The Reeb graph G1 is a quotient topology space including a topology, and a topological change occurs in the neighborhood of the value of f at a vertex.

The Reeb graph G1 has a graph structure that represents how the geometric structure (isopleth or iso-surface (Fermi surface)) composed of a point set having the same variable value topologically changes by changing the variable value. Thus, the Reeb graph G1 can represent a topological change related to the observed value 41b under each of the experimental conditions 41a in the plural times of experiments. Hence, it can be assumed that a characteristic of the topology changes with a vertex (black dot) on the Reeb graph G1 as a boundary. In other words, unless the value at a vertex on the Reeb graph G1 is exceeded, the topology does not change.

For example, how the number of Fermi surfaces in the wave number space (topology) changes by changing the value of f=z (experimental condition 41a) can readily be seen from the Reeb graph G1.

To be specific, the graph data generation unit 52 projects data on a lattice point in the multidimensional space with each of the experimental conditions 41a and the observed values 41b being a dimension, for the experimental data 41. Then, the graph data generation unit 52 triangulates the lattice (mesh) on which the data is projected. Then, the graph data generation unit 52 calculates the Reeb graph G1 by using a known calculation technique, such as Parsa algorithm, with the observed value 41b (for example, spectrum intensity) as a variable, for the triangulated mesh.

As an example, the graph data generation unit 52 sorts the vertices of the mesh in ascending order according to the values of f (experimental conditions 41a). Then, the graph data generation unit 52 calculates contours in the neighborhood of each of the larger and smaller values at each vertex. At this time, if the numbers of the contours are different, the graph data generation unit 52 adds a node to the Reeb graph G1 and inserts an edge at the node corresponding to that contour.

This Reeb graph G1 representing the correspondences between the experimental conditions 41a and the observed values 41b enables an at-a-glance view of a global change in the topology of the observed values 41b with different experimental conditions 41a.

The display data generation unit 53 is a processing unit that generates the display data (Fermi surface display data 43) indicating the experimental condition 41a and the observed value 41b corresponding to a specific point contained in the generated graph data 42 among the experimental conditions 41a and the observed values 41b included in the experimental data 41. To be specific, the display data generation unit 53 generates the Fermi surface display data 43 displaying the Fermi surface, corresponding to the specific point on the Reeb graph G1 of the graph data 42, in the wave number space under the experimental condition 41a and stores the Fermi surface display data 43 in the storage unit 40. This allows the control unit 50 to read out and display the graph data 42 on the display unit 30 to present the Fermi surface corresponding to the specific point on the Reeb graph G1 to the user.

To be specific, when the Reeb graph G1 of the graph data 42 is displayed on the display unit 30, the display data generation unit 53 receives specification of a point on the Reeb graph G1 through operation input by the user via the input unit 20. The display data generation unit 53 reads out a pair of the experimental condition 41a and the observed value 41b corresponding to the specified point and generates the Fermi surface display data 43 related to the Fermi surface corresponding to that point.

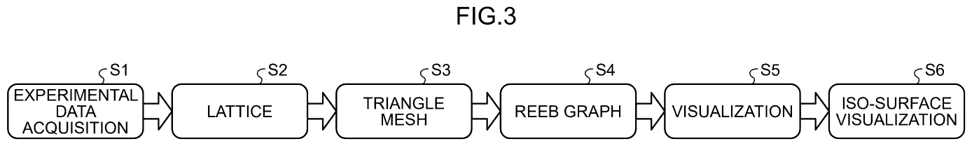

FIG. 3 is a flowchart illustrating example operation of the information processing device 1 according to the embodiment. As illustrated in FIG. 3, the experimental data acquisition unit 51 acquires experimental data 41 via the communication unit 10 or the input unit 20 (S1).

Then, the graph data generation unit 52 generates a lattice in a multidimensional space with each of the experimental conditions 41a and the observed values 41b being a dimension, for the acquired experimental data 41 (S2). Then, the graph data generation unit 52 projects the experimental data 41 on a lattice point and triangulates the lattice (mesh) to create a triangle mesh (S3).

Then, the graph data generation unit 52 calculates (generates) a Reeb graph (Reeb graph G1) by using a known calculation technique with the observed value 41b (for example, spectrum intensity) as a variable, for the triangle mesh (S4).

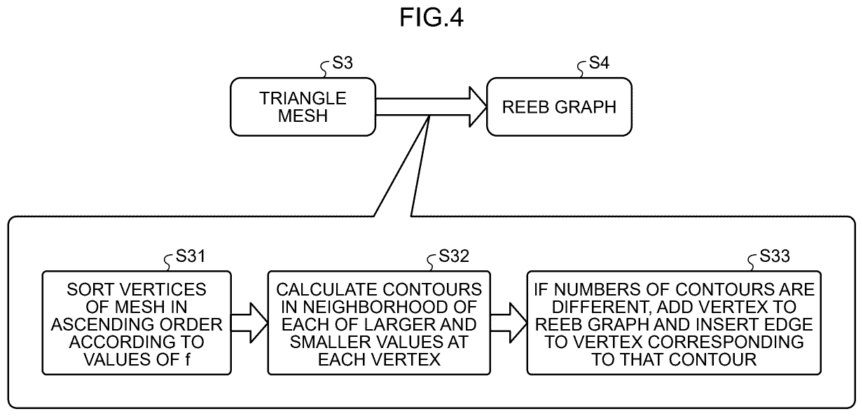

FIG. 4 is a flowchart illustrating an example process of generating the Reeb graph. As illustrated in FIG. 4, the graph data generation unit 52 sorts the vertices of the mesh in ascending order according to the values of f (experimental conditions 41a) (S31). Then, the graph data generation unit 52 calculates contours in the neighborhood of each of the larger and smaller values at each vertex (S32).

Then, if the numbers of the contours are different, the graph data generation unit 52 adds a node to the Reeb graph G1 and inserts an edge (edge) to the vertex (node) corresponding to that contour (S33).

Returning to FIG. 3, the control unit 50 reads out and displays the graph data 42 generated by the graph data generation unit 52 on the display unit 30 to visualize the Reeb graph G1 (S5).

Then, when the Reeb graph G1 is visualized, the display data generation unit 53 receives specification of a point on the Reeb graph G1 from the user. Then, the display data generation unit 53 reads out a pair of the experimental condition 41a and the observed value 41b corresponding to the specified point and generates the Fermi surface display data 43 related to the Fermi surface (iso-surface) corresponding to that point. The control unit 50 reads out and displays the Fermi surface display data 43 generated by the display data generation unit 53 on the display unit 30 to visualize the Fermi surface (iso-surface) (S6).

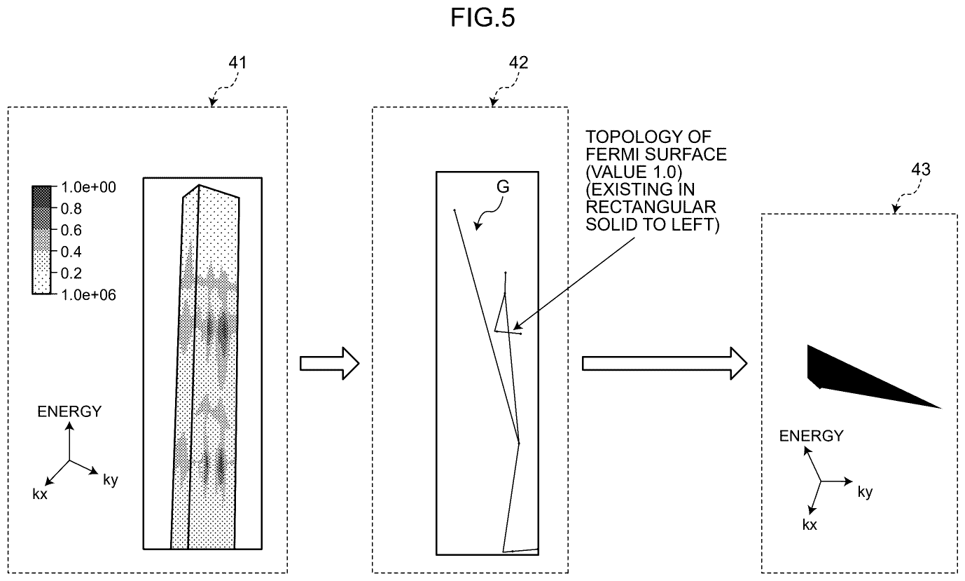

FIG. 5 is an explanatory diagram describing example display of the Reeb graph and the Fermi surface. As illustrated in FIG. 5, the experimental data 41 visualized by three-dimensionally plotting the experimental conditions 41a (energy) and the observed values 41b (wave number space (kx,ky)) has many hidden portions. Thus, even if the visualized experimental data 41 is visually observed, it is difficult to globally see how the topology of the Fermi surface (iso-surface) changes.

In contrast, with the graph data 42 visualizing the Reeb graph G1, it can readily be recognized how the Fermi surface changes topologically. Furthermore, from the Fermi surface display data 43 when a point on the Fermi surface (value 1.0) on the Reeb graph G1 is specified, the shape of the Fermi surface can readily be seen.

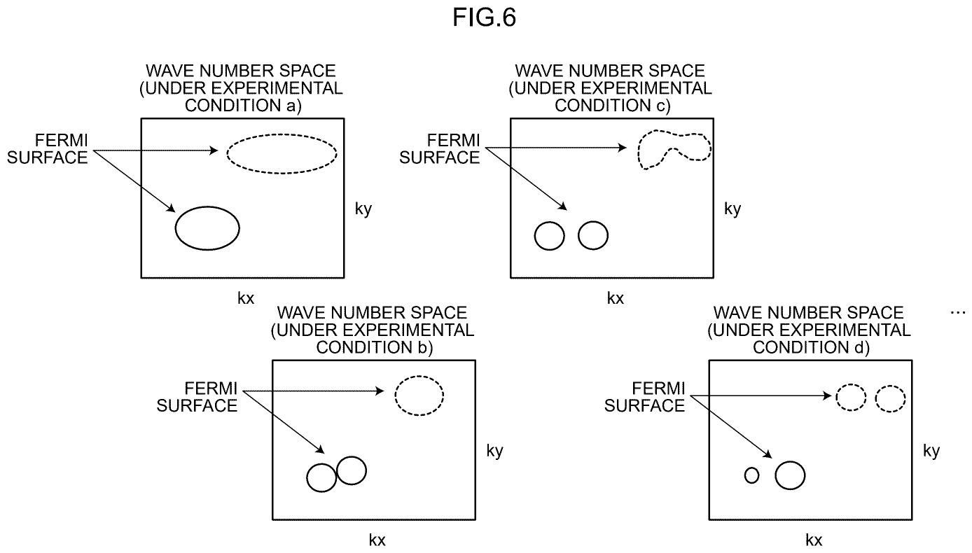

FIG. 6 is an explanatory diagram exemplifying the Fermi surface for each experimental condition. As illustrated in FIG. 6, it is difficult to follow how the topology of the Fermi surface changes by changing the experimental conditions throughout the experiment on the basis of a large number of experimental conditions (a, b, c, d, . . . ) existing and observed values (kx,ky) acquired under those experimental conditions. In contrast, the information processing device 1 presents the graph data 42 and the Fermi surface display data 43 to the user (See FIG. 5), so that the user can readily see how the topology of the Fermi surface changes.

As described above, the information processing device 1 acquires the experimental data 41 including the experimental condition 41a of each of the plural times of experiments and the observed value 41b under the experimental condition 41a. The information processing device 1 generates the graph data 42 indicating the topological structure of the geometric figure representing the correspondences between the experimental conditions 41a and the observed values 41b included in the experimental data 41 in the multidimensional space with each of the experimental conditions 41a and the observed values 41b being a dimension.

This enables the information processing device 1 to readily analyze, from the graph data 42, the topological structure of the geometric figure representing the correspondences between the experimental conditions 41a of the plural times of experiments and the observed values 41b, that is, how the topology of the observed values 41b changes by changing the experimental conditions 41a.

For example, from the experimental data 41 in physical property experiments with the intensity of electron energy applied to a metal surface as the experimental conditions 41a and information related to a Fermi surface in wave number space (an iso-surface in wave number space) as the observed values 41b, the topological structure of the Fermi surface can readily be analyzed. From the experimental data 41 in fluid experiments with the speed of a fluid as the experimental conditions 41a and streamlines as the observed values 41b, the topological structure (turbulent flow, laminar flow) of the streamlines can readily be analyzed. From the experimental data 41 in chemical experiments with temperature, humidity, and the like as the experimental conditions 41a and the amounts of a predetermined product generated chemically/biotically and the like as the observed values 41b, a search for a condition under which the predetermined product is generated and the like can readily be conducted.

Furthermore, the information processing device 1 generates the display data (Fermi surface display data 43) indicating the experimental condition 41a and the observed value 41b corresponding to a specific point contained in the generated graph data 42 among the experimental conditions 41a and the observed values 41b included in the experimental data 41. Thus, with the information processing device 1, from the specific point contained in the graph data 42, the experimental condition 41a and the observed value 41b corresponding to that point can readily be seen.

Furthermore, the graph data 42 is data indicating the Reeb graph G1 corresponding to the topological structure. This enables the information processing device 1 to analyze the topological structure by using the Reeb graph G1.

Note that each constituent of each device illustrated in the drawings does not exactly need to be physically configured as illustrated in the drawings. That is, the specific mode of dispersion/integration of each device is not limited to those illustrated in the drawings, and all or part thereof can be configured in a functionally or physically dispersed/integrated manner in optional units in accordance with various loads, usage conditions, and the like.

Various processing functions of the experimental data acquisition unit 51, the graph data generation unit 52, and the display data generation unit 53 performed by the control unit 50 of the information processing device 1 may be executed on a CPU (or a microcomputer, such as an MPU or a micro controller unit (MCU)) in whole or in optional part. Needless to say, the various processing functions may be executed on a computer program analyzed and executed on a CPU (or a microcomputer, such as an MPU or an MCU) or hardware using wired logic in whole or in optional part. Various processing functions performed by the information processing device 1 may be executed through cooperation of a plurality of computers by cloud computing.

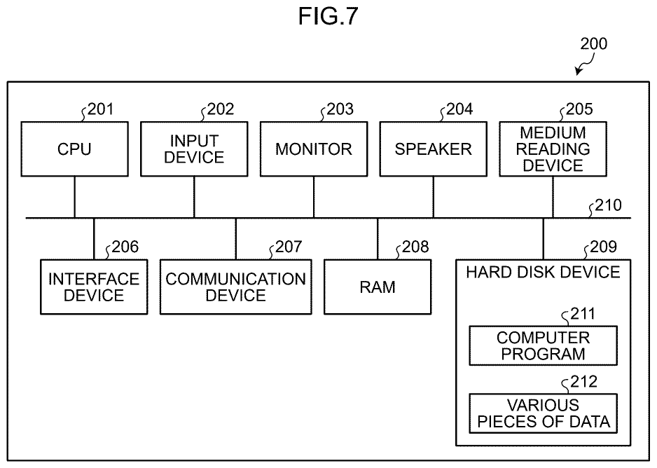

Various processes described in the above embodiments can be implemented by executing a preliminarily prepared computer program on a computer. An example configuration (hardware) of a computer executing a computer program having functions similar to the above embodiments will be described below. FIG. 7 is an explanatory diagram describing an example computer configuration.

As illustrated in FIG. 7, the computer 200 includes a CPU 201 that executes various arithmetic processes, an input device 202 that receives data input, a monitor 203, and a speaker 204. The computer 200 also includes a medium reading device 205 that reads a computer program and the like from a storage medium, an interface device 206 for connection to various devices, and a communication device 207 for wired or wireless communication connection to an external device. The computer 200 also includes a RAM 208 that temporarily stores various pieces of information therein and a hard disk device 209. Each constituent (201 to 209) of the computer 200 is connected to a bus 210.

The hard disk device 209 stores therein a computer program 211 for executing various processes in the functional configuration (for example, the experimental data acquisition unit 51, the graph data generation unit 52, and the display data generation unit 53) described in the above embodiments. The hard disk device 209 also stores therein various pieces of data 212 referenced by the computer program 211. The input device 202 receives, for example, input of operation information from an operator. The monitor 203 displays, for example, various screens operated by the operator. The interface device 206 is connected, for example, to a printing device and the like. The communication device 207 is connected to a communication network, such as a local area network (LAN) and exchanges various pieces of information with the external device via the communication network.

The CPU 201 reads out the computer program 211 stored in the hard disk device 209 and expands and executes the computer program 211 on the RAM 208 to perform various processes related to the above functional configuration (for example, the experimental data acquisition unit 51, the graph data generation unit 52, and the display data generation unit 53). Note that the computer program 211 does not need to be stored in the hard disk device 209. For example, the computer program 211 stored in a storage medium readable by the computer 200 may be read out and executed. The storage media readable by the computer 200 include, for example, a portable recording medium, such as a CD-ROM, a DVD disk, and a universal serial bus (USB) memory, a semiconductor memory such as a flash memory, hard disk drive, and the like. Alternatively, this computer program 211 may be stored in a device connected to a public network, the Internet, a LAN, or the like, and the computer 200 may read out the computer program 211 from these and execute the computer program 211.

According to embodiments, experimental analysis can be supported.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiment(s) of the present invention has (have) been described in detail, it should be understood that the various changes, substitutions, and alterations could be made hereto without departing from the spirit and scope of the invention.

What is claimed is:

1. A non-transitory computer-readable recording medium having stored therein a program that causes a computer to execute an information process comprising:
acquiring experimental data including an experimental condition of each of a plurality of times of experiments and an observed value under the experimental condition; and
generating graph data indicating a topological structure of a geometric figure representing correspondences between a plurality of the experimental conditions and the observed values in a multidimensional space with each of the experimental conditions and the observed values being a dimension, the experimental conditions and the observed values being included in the experimental data.

2. The non-transitory computer-readable recording medium according to claim 1, wherein the program causes the computer to further execute an information process that generates display data indicating an experimental condition and an observed value among the experimental conditions and the observed values included in the experimental data, the experimental condition and the observed value corresponding to a specific point contained in the generated graph data.

3. The non-transitory computer-readable recording medium according to claim 1, wherein the graph data is data indicating a Reeb graph corresponding to the topological structure.

4. An information processing method comprising:
causing a computer to execute a process including
acquiring experimental data including an experimental condition of each of a plurality of times of experiments and an observed value under the experimental condition, and
generating graph data indicating a topological structure of a geometric figure representing a correspondence between the experimental condition and the observed value in a multidimensional space with each of the experimental condition and the observed value being a dimension.

5. The information processing method according to claim 4, wherein the computer further executes a process that generates display data indicating an experimental condition and an observed value among a plurality of the experimental conditions and the observed values included in the experimental data, the experimental condition and the observed value corresponding to a specific point contained in the generated graph data.

6. The information processing method according to claim 4, wherein the graph data is data indicating a Reeb graph corresponding to the topological structure.

7. An information processing device comprising:
a control unit that executes a process including
acquiring experimental data including an experimental condition of each of a plurality of times of experiments and an observed value under the experimental condition, and
generating graph data indicating a topological structure of a geometric figure representing a correspondence between the experimental condition and the observed value in a multidimensional space with each of the experimental condition and the observed value being a dimension.

8. The information processing device according to claim 7, wherein the control unit further executes a process that generates display data indicating an experimental condition and an observed value among a plurality of the experimental conditions and the observed values included in the experimental data, the experimental condition and the observed value corresponding to a specific point contained in the generated graph data.

9. The information processing device according to claim 7, wherein the graph data is data indicating a Reeb graph corresponding to the topological structure.
